(19) United States
(12) Patent Application Publication
(10) Pub. No.: US 2003/0065043 A1
(43) Pub. Date: Apr. 3, 2003

(54) Promoted catalysts and fischer-tropsch processes

(75) Inventors: Olga P. Ionkina, Ponca City, OK (US); Kamel M. Makar, Wilmington, DE (US); Leo E. Manzer, Wilmington, DE (US); Munirpallam A. Subramanian, Kennett Square, PA (US)

Correspondence Address: DAVID W. WESTPHAL, CONOCO PHILLIPS, P.O. BOX 1267, PONCA CITY, OK, 74602-1267, US

(73) Assignee: Conoco Inc., Houston, TX

(21) Appl. No.: 10/230,496

(22) Filed: Aug. 29, 2002

Related U.S. Application Data
(60) Provisional application No. 60/316,826, filed on Aug. 31, 2001.

(51) Int. Cl. C07C 27/06

(52) U.S. Cl. 518/714

(57) ABSTRACT

A process is disclosed for producing hydrocarbons. The process involves contacting a feed stream comprising hydrogen and carbon monoxide with a catalyst in a reaction zone maintained at conversion-promoting conditions effective to produce an effluent stream comprising hydrocarbons. In accordance with this invention, the catalyst used in the process includes at least a Fischer-Tropsch metal and a promoter selected from the group consisting of molybdenum, tin, gallium, and zinc. The Fischer-Tropsch metal preferably includes cobalt. The catalyst may also include a support material selected from the group including silica, titania, titania/alumina, zirconia, alumina, silica-alumina, aluminum fluoride, and fluorided aluminas.

### CROSS-REFERENCE TO RELATED APPLICATIONS

The present application claims the benefit of 35 U.S.C. 111(b) provisional application Serial No. 60/316,826 filed Aug. 31, 2001, and entitled Promoted Catalysts and Fischer-Tropsch Processes.

### STATEMENT REGARDING FEDERALLY SPONSORED RESEARCH OR DEVELOPMENT

Not applicable.

### FIELD OF THE INVENTION

The present invention relates to a process for the preparation of hydrocarbons from synthesis gas, (i.e., a mixture of carbon monoxide and hydrogen), typically labeled the Fischer-Tropsch process. Particularly, this invention relates to the use of supported catalysts containing a Fischer-Tropsch catalytic metal, preferably cobalt and a promoter selected from the group consisting of molybdenum, tin, gallium, and zinc for the Fischer-Tropsch process.

### BACKGROUND OF THE INVENTION

Large quantities of methane, the main component of natural gas, are available in many areas of the world. Methane can be used as a starting material for the production of hydrocarbons. The conversion of methane to hydrocarbons is typically carried out in two steps. In the first step methane is reformed with water or partially oxidized with oxygen to produce carbon monoxide and hydrogen (i.e., synthesis gas or syngas). In a second step, the syngas is converted to hydrocarbons. This second step, the preparation of hydrocarbons from synthesis gas is well known in the art and is usually referred to as Fischer-Tropsch synthesis, the Fischer-Tropsch process, or Fischer-Tropsch reaction(s).

The Fischer-Tropsch reaction involves the catalytic hydrogenation of carbon monoxide to produce a variety of products ranging from methane to higher aliphatic alcohols. The process has been considered for the conversion of carbonaceous feedstock, e.g., coal or natural gas, to higher value liquid fuel or petrochemicals. The methanation reaction was first described in the early 1900's, and the later work by Fischer and Tropsch dealing with higher hydrocarbon synthesis was described in the 1920's. The first major commercial use of the Fischer-Tropsch process was in Germany during the 1930's. More than 10,000 B/D (barrels per day) of products were manufactured with a cobalt based catalyst in a fixed-bed reactor. This work has been described by Fischer and Pichler in Ger. Pat. No. 731,295 issued Aug. 2, 1936, hereby incorporated herein by reference. Commercial practice of the Fischer-Tropsch process has continued from 1954 to the present day in South Africa in the SASOL plants. These plants use iron-based catalysts, and produce gasoline in relatively high-temperature fluid-bed reactors and wax in relatively low-temperature fixed-bed reactors.

The Fischer-Tropsch synthesis reactions are highly exothermic and reaction vessels must be designed for adequate heat exchange capacity. Because the feed streams to Fischer-Tropsch reaction vessels are gases while the product streams include liquids, the reaction vessels must have the ability to continuously produce and remove the desired range of liquid hydrocarbon products. Motivated by production of high-grade gasoline from natural gas, research on the possible use of the fluidized bed for Fischer-Tropsch synthesis was conducted in the United States in the mid-1940s. Based on laboratory results, Hydrocarbon Research, Inc. constructed a dense-phase fluidized bed reactor, the Hydrocol unit, at Carthage, Tex., using powdered iron as the catalyst. Due to disappointing levels of conversion, scale-up problems, and rising natural gas prices, operations at this plant were suspended in 1957. Research has continued, however, on developing Fischer-Tropsch reactors such as slurry-bubble columns, as disclosed in U.S Pat. No. 5,348,982 issued Sep. 20, 1994, hereby incorporated herein by reference.

Catalysts for use in the Fischer-Tropsch synthesis usually contain a catalytically active metal of Groups 8, 9, 10 (in the New notation of the periodic table of the elements, which is followed throughout). In particular, iron, cobalt, nickel, and ruthenium, and combinations thereof, have been abundantly used as the catalytically active metals. Cobalt and ruthenium have been found to be particularly suitable for catalyzing a process in which synthesis gas is converted to primarily hydrocarbons having five or more carbon atoms (i.e., where the C₅₊ selectivity of the catalyst is high). However, due to its expense and rarity, ruthenium is typically used in combination with another of the catalytically active metals, such as cobalt. For example, U.S. Pat. No. 4,088,671, hereby incorporated herein by reference, discloses a process for the synthesis of higher hydrocarbons from the reaction of CO and hydrogen at low pressure in the contact presence of a catalyst comprising as the active ingredients a major amount of cobalt and a minor amount of ruthenium.

Additionally, the catalysts often contain a support or carrier material. Supports for catalysts used in Fischer-Tropsch synthesis of hydrocarbons have typically been refractory oxides (e.g., silica, alumina, titania, zirconia or mixtures thereof, such as silica-alumina). A support may be used to provide a high surface area for contact of the catalytically active metal with the syngas, to reduce the amount of catalytically active metal used, or to otherwise improve the performance or economics of catalysts and catalytic processes.

Additionally, Fischer-Tropsch catalysts often contain one or more promoters. For example, promoters that have been used for cobalt-ruthenium catalysts include thorium, lanthanum, magnesium, manganese, and rhenium. A promoter may have any of various desirable functions, such as improving activity, productivity, selectivity, lifetime, regenerability, or other properties of catalysts and catalytic processes.

There are significant differences in the molecular weight distributions of the hydrocarbon products from Fischer-Tropsch reaction systems. Product distribution or product selectivity depends heavily on the type and structure of the catalysts and on the reactor type and operating conditions. Accordingly, it is highly desirable to maximize the selectivity of the Fischer-Tropsch synthesis to the production of high-value liquid hydrocarbons, such as hydrocarbons with five or more carbon atoms per hydrocarbon chain.

Research is continuing on the development of more efficient Fischer-Tropsch catalyst systems and reaction systems that increase the selectivity for high-value hydrocarbons in the Fischer-Tropsch product stream. High value hydrocarbons include those useful for further processing to yield gasoline, for example C₅₊ hydrocarbons, particularly C₅₋-C₁₀ hydrocarbons, and those useful for further processing to yield diesel fuel, for example C₁₁₊ hydrocarbons, particularly C₁₁-C₂₀ hydrocarbons. A common way to measure the overall selectivity of the Fischer-Tropsch products is the chain growth probability of alpha value. The higher the alpha value, the higher the selectivity towards C₅₊ hydrocarbons and therefore, the lower the selectivity towards C₄₋ hydrocarbons. A number of studies describe the behavior of iron, cobalt or ruthenium based catalysts in various reactor types, together with the development of catalyst compositions and preparations. For example, see the articles “Short history and present trends of Fischer-Tropsch synthesis,” by H. Schlutz, Applied Catalysis A 186, 3-12, 1999, and “Status and future opportunities for conversion of synthesis gas to liquid fuels, by G. Alex Mills, Fuel 73, 1243-1279, 1994, each hereby incorporated herein by reference in their entirety.

Notwithstanding the above teachings, it continues to be desirable to improve the activity and reduce the cost of Fischer-Tropsch catalysts and processes. In particular, there is still a great need to identify new promoted catalysts useful for Fischer-Tropsch synthesis, particularly catalysts that provide high C₁₁₊ hydrocarbon selectivities to maximize the value of the hydrocarbons produced and thus the process economics.

### SUMMARY OF THE INVENTION

This invention provides a process and catalyst for producing hydrocarbons, and a method for preparing the catalyst. The process comprises contacting a feed stream comprising hydrogen and carbon monoxide with a catalyst in a reaction zone maintained at conversion-promoting conditions effective to produce an effluent stream comprising hydrocarbons.

According to an embodiment of the present invention, the catalyst used in the process comprises a promoter selected from the group consisting of molybdenum, tin, gallium, and zinc and a Fischer-Tropsch metal. The Fischer-Tropsch metal preferably includes cobalt.

According to another embodiment of the present invention, a method for the preparation of a supported Fischer-Tropsch catalyst includes supporting a promoter selected from the group consisting of molybdenum, tin, gallium, and zinc and cobalt on a support material selected from the group including silica, titania, titania/alumina, zirconia, alumina, silica-alumina, aluminum fluoride, and fluorided alumina.

According to still another embodiment of the present invention a process for producing hydrocarbons includes contacting a feed stream comprising hydrogen and carbon monoxide with a supported catalyst in a reaction zone maintained at conversion-promoting conditions effective to produce an effluent stream comprising hydrocarbons. The catalyst used in the process includes a promoter selected from the group consisting of molybdenum, tin, gallium, and zinc and cobalt. The catalyst may further include a support selected from the group including silica, titania, titania/alumina, zirconia, alumina, silica-alumina, borated alumina, aluminum fluoride, and fluorided aluminas.

The above-described process for producing hydrocarbon may be characterized by a measure of activity for the production of hydrocarbons having a weight range of at least the middle distillate range that is higher than that for a corresponding process comprising contact a feed stream comprising hydrogen and carbon monoxide with a corresponding unpromoted catalyst in a reaction zone maintained at conversion-promoting conditions effective to produce an effluent stream comprising hydrocarbons. The measure of activity may be the productivity. Alternatively the measure of activity may be the selectivity.

Further, the catalyst used in the above-described process for producing hydrocarbons may include not more than 5000 ppm of precious metal promoter. The precious metal promoter is preferably selected from the group consisting of rhenium, ruthenium and platinum.

### DETAILED DESCRIPTION OF THE PREFERRED EMBODIMENTS

The present catalyst contains a catalytically effective amount of a Fischer-Tropsch metal. The amount of metal present in the catalyst may vary widely. Typically, when the catalyst includes a support, the catalyst comprises from about 1 to 50% by weight (as the metal) of the total supported metal per total weight of catalytic metal and support, preferably from about 5 to 40% by weight, and more preferably from about 10 to 35% by weight. A Fischer-Tropsch metal may include an element selected from among a Group 8 element (e.g. Fe, Ru, and Os), a Group 9 element (e.g. Co, Rh, and Ir), a Group 10 element (e.g. Ni, Pd, and Pt), and combinations thereof. Preferably, the Fischer-Tropsch metal includes cobalt.

We have found that higher selectivity and productivity catalysts are produced when a promoter selected from the group consisting of molybdenum, tin, gallium, and zinc is added to the catalyst. Productivities in batch testing can equal or exceed 400 g/hr/kg-cat for molybdenum and tin, an increase with respect to the productivity observed for a comparative unpromoted cobalt catalyst. Further, the productivity increase with respect to the corresponding unpromoted catalyst can equal or exceed 50%. Likewise, the chain growth probability or a, can each equal or exceed 0.9 for gallium and zinc, an increase with respect to an unpromoted catalyst. Further, the present inventors have found that batch testing results such as these for improved performance of a promoted catalyst with respect to the corresponding unpromoted catalyst tend to be predictive of corresponding improved performance in other reaction environments, such as continuous operation. An advantage of the present invention is that an improved performance as compared with an unpromoted catalyst is achieved in the absence of precious metal promoters such as rhenium, ruthenium, platinum, and the like.

Improved performance of the present catalysts is preferably indicated by an increase of a measure of the activity for the production of hydrocarbons having a weight range at least the middle distillate weight range, such as C₁₁⁺ hydrocarbons, as compared to the activity for the production of hydrocarbons of a corresponding unpromoted catalyst. The measure of activity may be the productivity or selectivity, wherein the selectivity may be indicated by the value of α.

The amount of promoter is added to the catalyst in a concentration sufficient to provide a weight ratio of elemental promoter:elemental catalytic metal of from about 0.00005:1 to about 0.5:1, preferably, from about 0.0005:1 to about 0.01:1 (dry basis).

The present catalyst material may be supported on any suitable support. Supports that are contemplated for use with a catalyst according to the preferred embodiments of the present invention include silica, titania, titania/alumina, zirconia, alumina, silica, titania, titania/alumina, and the like. Further, suitable supports include those disclosed in co-pending commonly assigned U.S. patent applications Ser. No. 09/314,921, Attorney Docket Number 1856-00600, entitled “Fischer-Tropsch Catalysts and Processes Using Fluorided Supports, Ser. No. 09/314,920, Attorney Docket Number 1856-00700, entitled “Fischer-Tropsch Processes and Catalysts Using Fluorided Alumina Supports”, and Ser. No. 60/215,718, Attorney Docket Number 1856-08000, entitled “Fischer-Tropsch Processes and Catalysts Using Aluminum Borate Supports”, each hereby incorporated herein by reference.

The catalysts of the preferred embodiments of the present invention may be prepared by any of the methods known to those skilled in the art. By way of illustration and not limitation, methods for preparing supported catalysts include impregnating the catalytically active compounds or precursors onto a support in one or more steps, extruding one or more catalytically active compounds or precursors together with support material to prepare catalyst extrudates, and/or precipitating the catalytically active compounds or precursors onto a support. Accordingly, supported catalysts according to a preferred embodiment of the present invention may be used in the form of powders, particles, pellets, monoliths, honeycombs, packed beds, foams, and aerogels.

The most preferred method of preparation may vary among those skilled in the art, depending for example on the desired catalyst particle size. Those skilled in the art are able to select the most suitable method for a given set of requirements.

One method of preparing a supported metal catalyst such as a supported cobalt-containing catalyst is by incipient wetness impregnation of the support with an aqueous solution of a soluble metal salt such as nitrate, acetate, acetylacetonate or the like. Another method of preparing a supported metal catalyst is by a melt impregnation technique, which involves preparing the supported metal catalyst from a molten metal salt. One preferred method is to impregnate the support with a molten metal nitrate (e.g., Co(NO₃)₂.6H₂O). Alternatively, the support can be impregnated with a solution of a zero valent metal precursor. One preferred method is to impregnate the support with a solution of zero valent cobalt such as Co₂(CO)₈, Co₄(CO)₁₂ or the like in a suitable organic solvent (e.g., toluene).

The most preferred sequence of addition of elements to a support may vary among those skilled in the art. For example, it is contemplated that the Fischer-Tropsch metal and a promoter may be added to a support in the same mixture. Alternatively, the Fischer-Tropsch metal and the promoter may be added in separate steps. Thus a supported catalyst according to a preferred embodiment of the present invention may include co-dispersed Fischer-Tropsch metal and a promoter. Alternatively, a supported catalyst according to a preferred embodiment of the present invention may include a layer containing a Fischer-Tropsch metal and a layer containing a promoter selected from the group consisting of molybdenum, tin, gallium, and zinc.

The impregnated support is dried and reduced with hydrogen or a hydrogen containing gas. The hydrogen reduction step may not be necessary if the catalyst is prepared with zero valent cobalt. In another preferred method, the impregnated support is dried, and calcined in the presence of air or oxygen and reduced in the presence of a hydrogen-containing gas.

Typically, at least a portion of the metal(s) of the catalytic metal component (a) of the catalysts of the present invention is present in a reduced state (i.e., in the metallic state). Therefore, it is normally advantageous to activate the catalyst prior to use by a reduction treatment, in the presence of hydrogen at an elevated temperature. Typically, the catalyst is treated with hydrogen at a temperature in the range of from about 75° C. to about 500° C., for about 0.5 to about 36 hours at a pressure of about 1 to about 75 atm. Pure hydrogen may be used in the reduction treatment, as may a mixture of hydrogen and an inert gas such as nitrogen, or a mixture of hydrogen and other gases as are known in the art, such as carbon monoxide and carbon dioxide. Reduction with pure hydrogen and reduction with a mixture of hydrogen and carbon monoxide are preferred. The amount of hydrogen may range from about 1% to about 100% by volume.

The catalysts of the preferred embodiments of the present invention are preferably used in a catalytic process for production of hydrocarbons, most preferably the Fischer-Tropsch process. The feed gases charged to the process of the preferred embodiment of the present invention comprise hydrogen, or a hydrogen source, and carbon monoxide. H₂/CO mixtures suitable as a feedstock for conversion to hydrocarbons according to the process of this invention can be obtained from light hydrocarbons such as methane by means of steam reforming, partial oxidation, or other processes known in the art. Preferably the hydrogen is provided by free hydrogen, although some Fischer-Tropsch catalysts have sufficient water gas shift activity to convert some water to hydrogen for use in the Fischer-Tropsch process. It is preferred that the molar ratio of hydrogen to carbon monoxide in the feed be greater than 0.5:1 (e.g., from about 0.67 to 2.5). Preferably, the feed gas stream contains hydrogen and carbon monoxide in a molar ratio of about 1.8:1 to 2.3:1. The feed gas may also contain carbon dioxide. The feed gas stream could contain a low concentration of compounds or elements that have a deleterious effect on the catalyst, such as poisons. For example, the feed gas may need to be pre-treated to ensure that it contains low concentrations of sulfur or nitrogen compounds such as hydrogen sulfide, ammonia and carbonyl sulfides.

The feed gas is contacted with the catalyst in a reaction zone. Mechanical arrangements of conventional design may be employed as the reaction zone including, for example, fixed bed, fluidized bed, slurry phase, slurry bubble column, reactive distillation column, or ebullating bed reactors, among others, may be used. Accordingly, the size and physical form of the catalyst particles may vary depending on the reactor in which they are to be used.

The Fischer-Tropsch process is typically run in a continuous mode. In this mode, the gas hourly space velocity through the reaction zone typically may range from about 50 volumes/hour/reactor volume (v/hr/v) to about 10,000 v/hr/v, preferably from about 300 v/hr/v to about 2,000 v/hr/v. The reaction zone temperature is typically in the range from about 160° C. to about 300° C. Preferably, the reaction zone is operated at conversion promoting conditions at temperatures from about 190° C. to about 260° C. The reaction zone pressure is typically in the range of about 80 psia (552 kPa) to about 1000 psia (6895 kPa), more preferably from 80 psia (552 kPa) to about 600 psia (4137 kPa), and still more preferably, from about 140 psia (965 kPa) to about 500 psia (3447 kPa).

The products resulting from the process will have a great range of molecular weights. Typically, the carbon number range of the product hydrocarbons will start at methane and continue to the limits observable by modern analysis, about 50 to 100 carbons per molecule. The process is particularly useful for making hydrocarbons having five or more carbon atoms especially when the above-referenced preferred space velocity, temperature and pressure ranges are employed.

The wide range of hydrocarbons produced in the reaction zone will typically afford liquid phase products at the reaction zone operating conditions. Therefore the effluent stream of the reaction zone will often be a mixed phase stream including liquid and vapor phase products. The effluent stream of the reaction zone may be cooled to effect the condensation of additional amounts of hydrocarbons and passed into a vapor-liquid separation zone separating the liquid and vapor phase products. The vapor phase material may be passed into a second stage of cooling for recovery of additional hydrocarbons. The liquid phase material from the initial vapor-liquid separation zone together with any liquid from a subsequent separation zone may be fed into a fractionation column. Typically, a stripping column is employed first to remove light hydrocarbons such as propane and butane. The remaining hydrocarbons may be passed into a fractionation column where they are separated by boiling point range into products such as naphtha, diesel and heavier hydrocarbons. Hydrocarbons recovered from the reaction zone and having a boiling point above that of the desired products may be passed into conventional processing equipment such as a hydrocracking zone in order to reduce their molecular weight. The gas phase recovered from the reactor zone effluent stream after hydrocarbon recovery may be partially recycled if it contains a sufficient quantity of hydrogen and/or carbon monoxide.

Without further elaboration, it is believed that one skilled in the art can, using the description herein, utilize the present invention to its fullest extent. The following embodiments are to be construed as illustrative, and not as constraining the scope of the present invention in any way whatsoever. For example, it will be understood that while batch testing is described, a process for producing hydrocarbons may alternatively be operated in continuous mode.

### EXAMPLES

General Procedure for Melt Impregnation

For each of the examples, the cobalt precursor was melted and each of the other precursors was dissolved in a small amount of a suitable solvent and mixed well with the melted cobalt precursor to form a solution. The solvent for Ru(III)2,4-pentane-dionate was CH₃CN and the solvent for MoO₃ was nitric acid. The solvent for the other promoter precursors was water. The support was slurried into the mixture. Amounts and identities of the support and each of the precursors are indicated in Table 1. The catalyst of Example 4 is a comparative corresponding catalyst that is unpromoted. Therefore no precursor was used in making the catalyst of Example 4.

General Procedure for Treatment of Catalyst Preparation Slurry

Each of the catalyst preparation slurries resulting from slurrying a support into a solution containing a Fischer-Tropsch metal precursor, as described below, was dried at a drying temperature, typically 80° C. The solids were removed from the oven and exposed to air to absorb moisture. The solids were then dried again at the same drying temperature, typically 80° C., followed by heating the solids at 0.5° C. per minute to a calcination temperature, typically 350° C., and maintaining the solids at this temperature for 18 minutes. The solids were then heated at 0.5° C. per minute to 450° C., and reduced in hydrogen flow at 450° C. for 6 hours. The material was cooled and flushed with nitrogen overnight and then sealed for transport into an inert atmosphere glove box. The recovered catalyst was bottled and sealed for storage inside the glove box until Fischer-Tropsch testing could be completed.

General Procedure for Batch Testing

For the batch tests, a 2 mL pressure vessel was heated at 225° C. under 1000 psig (6994 kPa) of H₂:CO (2:1) and maintained at that temperature and pressure for 1 hour. In a typical run, roughly 20 mg of the reduced catalyst and 1 mL of n-octane was added to the vessel. After one hour, the reactor vessel was cooled in ice, vented, and an internal standard of di-n-butylether was added. The reaction product was analyzed on an HP6890 gas chromatograph. Hydrocarbons in the range of C₁₁-C₄₀ were analyzed relative to the internal standard. The lower hydrocarbons were not analyzed, since they are masked by the solvent and are also vented as the pressure is reduced.

A nominal composition was computed according to the amount by weight of alumina, the amount of elemental cobalt, and the amount of elemental promoter used to prepare the catalyst. Where a % is used in the nominal composition, it is a weight %.

A C₁₁₊ Productivity (g C₁₁₊ /hour/kg catalyst) was calculated based on the integrated production of the C₁₁-C₄₀ hydrocarbons per kg of catalyst per hour. The logarithm of the weight fraction for each carbon number, divided by the carbon number, ln(Wn/n) was plotted as the ordinate vs. number of carbon atoms in (Wₙ/n) as the abscissa. From the slope, a value of alpha was obtained. Results of batch testing under standard operating conditions of a temperature of about 225° C. and a pressure of about 1000 psi are summarized in Table 2.

The results listed in Table 2, Examples 1 and 2, show that the use of a catalyst that includes molybdenum, for example a catalyst a nominal composition including 1% molybdenum by weight, improves productivity with respect to a corresponding unpromoted catalyst (Example 4). Further, the results listed in Table 2, Examples 3 and 7, show that the use of a catalyst that includes an optimal amount of tin improves productivity with respect to a corresponding unpromoted catalyst (Example 4). For a catalyst with a nominal composition of 0.1% tin, activity was improved, whereas for a catalyst with a nominal composition of 0.5% tin, activity was reduced with respect to a corresponding unpromoted catalyst (Example 4). Still further, the results listed in Table 2, Examples 5 and 6 show that the use of a catalyst that includes gallium or zinc, respectively, improves selectivity with respect to a corresponding unpromoted catalyst (Example 4). This improvement in selectivity is demonstrated by an increase in the value of a. The improved selectivity is to hydrocarbons with a weight range of at least the middle distillate weight range.

TABLE 1

| | | Co(NO₃)₂• | | |
|---|---|---|---|---|
| | Al₂O₃ | 6H₂O | | wt. |
| Example No. | (gm) | (gm) | precursor | (gm) |
| 1 | 3.9500 | 4.9384 | MoO₃ | 0.0750 |
| 2 | 3.9500 | 4.9384 | MoO₃ | 0.0750 |
| 3 | 3.9950 | 4.9383 | SnCl₄• | 0.0148 |
| | | | 5H₂O | |
| 4 | 4.0000 | 4.9384 | none | 0 |
| 5 | 3.9750 | 4.9383 | Ga(NO₃)₃• | 0.1305 |
| | | | .6H₂O | |
| 6 | 3.9750 | 4.9383 | Zn(NO₃)₂• | 0.1137 |
| | | | 6H₂O | |
| 7 | 3.9750 | 4.9383 | SnCl₄• | 0.0738 |
| | | | 5H₂O | |

TABLE 2

| | | C_{11 +} | |
|---|---|---|---|
| Example No. | Catalyst Nominal Composition | Productivity | α |
| 1 | 20%Co/1%Mo/Al₂O₃ | 590 | 0.88 |
| 2 | 20%Co/1%Mo/Al₂O₃ | 590 | 0.89 |
| 3 | 20%Co/0.1%Sn/Al₂O₃ | 400 | 0.89 |
| 4 | 20%Co/Al₂O₃ | 380 | 0.88 |
| 5 | 20%Co/0.5%Ga/Al₂O₃ | 360 | 0.91 |
| 6 | 20%Co/0.5%Zn/Al₂O₃ | 320 | 0.9 |
| 7 | 20%Co/0.5%Sn/Al₂O₃ | 140 | 0.87 |

While a preferred embodiment of the present invention has been shown and described, it will be understood that variations can be made to the preferred embodiment without departing from the scope of, and which are equivalent to, the present invention. For example, the structure and composition of the catalyst can be modified and the process steps can be varied.

The complete disclosures of all patents, patent documents, and publications cited herein are hereby incorporated by reference in their entirety.

The foregoing detailed description and examples have been given for clarity of understanding only. No unnecessary limitations are to be understood therefrom. The invention is not limited to the exact details shown and described, for variations obvious to one skilled in the art will be included within the invention by the claims.

We claim:

1. A process for producing hydrocarbons, comprising contacting a feed stream comprising hydrogen and carbon monoxide with a catalyst in a reaction zone maintained at conversion-promoting conditions effective to produce an effluent stream comprising hydrocarbons; said catalyst comprising cobalt and a promoter comprising at least one element selected from the group consisting of molybdenum, tin, gallium, and zinc.

2. The process of claim 1 wherein the catalyst further comprises a support selected from the group consisting of silica, titania, titania/alumina, zirconia, alumina, silica-alumina, borated alumina, aluminum fluoride, and fluorided aluminas.

3. The process of claim 1 wherein the catalyst is prepared from a zero valent metal precursor.

4. The process of claim 1 wherein the catalyst is prepared from a molten metal salt.

5. The process of claim 1 characterized by a productivity for the production of hydrocarbons having a weight range of at least the middle distillate range that is higher than that for a corresponding process comprising contact a feed stream comprising hydrogen and carbon monoxide with a corresponding unpromoted catalyst in a reaction zone maintained at conversion-promoting conditions effective to produce an effluent stream comprising hydrocarbons.

6. The process of claim 5 wherein the promoter is selected from the group consisting of molybdenum and tin.

7. The process of claim 5 wherein the productivity is at least about 5% higher.

8. The process of claim 5 wherein the productivity is at least about 50% higher.

9. The process of claim 1 characterized by a selectivity for the production of hydrocarbons having a weight range of at least the middle distillate range that is higher than that for a corresponding process comprising contact a feed stream comprising hydrogen and carbon monoxide with a corresponding unpromoted catalyst in a reaction zone maintained at conversion-promoting conditions effective to produce an effluent stream comprising hydrocarbons.

10. The process of claim 9 wherein the promoter is selected from the group consisting of gallium and zinc.

11. The process of claim 9 wherein the hydrocarbons have a carbon number distribution described by a value of α of at least about 0.9.

12. The process of claim 9 wherein the catalyst further comprises up to 5000 ppm of one or more precious metal selected from the group consisting of rhenium, ruthenium, and platinum.

13. A process for producing hydrocarbons, comprising contacting a feed stream comprising hydrogen and carbon monoxide with a catalyst in a reaction zone maintained at conversion-promoting conditions effective to produce an effluent stream comprising hydrocarbons; said catalyst comprising cobalt and a promoter comprising at least one element selected from the group consisting of molybdenum, tin, gallium, and zinc, the process characterized by a measure of activity for the production of hydrocarbons having a weight range of at least the middle distillate range that is higher than that for a corresponding process comprising contact a feed stream comprising hydrogen and carbon monoxide with a corresponding unpromoted catalyst in a reaction zone maintained at conversion-promoting conditions effective to produce an effluent stream comprising hydrocarbons.

14. The process according to claim 13 wherein the measure of activity is the productivity.

15. The process according to claim 13 wherein the measure of activity is the selectivity.

16. The process according to claim 13 wherein the promoter comprises molybdenum.

17. The process according to claim 13 wherein the promoter comprises tin.

18. The process according to claim 13 wherein the promoter comprises gallium.

19. The process according to claim 13 wherein the promoter comprises zinc.

20. The process according to claim 13 wherein the catalyst comprises up to 5000 ppm of precious metal promoter.

21. The process according to claim 20 wherein the precious metal promoter is selected from the group consisting of ruthenium, platinum and rhenium.

22. The process of claim 13 wherein the catalyst further comprises a support selected from the group consisting of silica, titania, titania/alumina, zirconia, alumina, silica-alumina, borated alumina, aluminum fluoride, and fluorided aluminas.

23. A process for producing hydrocarbons, comprising contacting a feed stream comprising hydrogen and carbon monoxide with a catalyst in a reaction zone maintained at conversion-promoting conditions effective to produce an effluent stream comprising hydrocarbons; said catalyst comprising cobalt and a promoter comprising at least one element selected from the group consisting of molybdenum, tin, gallium, and zinc wherein the catalyst comprises not more than a trace amount of a precious metal promoter selected from the group consisting of ruthenium, platinum and rhenium.

24. The process of claim 23 wherein the catalyst further comprises a support selected from the group consisting of silica, titania, titania/alumina, zirconia, alumina, silica-alumina, borated alumina, aluminum fluoride, and fluorided aluminas.

25. The process of claim 23 wherein the catalyst is prepared from a zero valent metal precursor.

26. The process of claim 23 wherein the catalyst is prepared from a molten metal salt.

27. The process of claim 23 characterized by a productivity for the production of hydrocarbons having a weight range of at least the middle distillate range that is higher than that for a corresponding process comprising contact a feed stream comprising hydrogen and carbon monoxide with a corresponding unpromoted catalyst in a reaction zone maintained at conversion-promoting conditions effective to produce an effluent stream comprising hydrocarbons.

28. The process of claim 27 wherein the promoter is selected from the group consisting of molybdenum and tin.

29. The process of claim 27 wherein the productivity is at least about 5% higher.

30. The process of claim 27 wherein the productivity is at least about 50% higher.

31. The process of claim 23 characterized by a selectivity for the production of hydrocarbons having a weight range of at least the middle distillate range that is higher than that for a corresponding process comprising contact a feed stream comprising hydrogen and carbon monoxide with a corresponding unpromoted catalyst in a reaction zone maintained at conversion-promoting conditions effective to produce an effluent stream comprising hydrocarbons.

32. The process of claim 31 wherein the promoter is selected from the group consisting of gallium and zinc.

33. The process of claim 31 wherein the hydrocarbons have a carbon number distribution described by a value of α of at least about 0.9.
